(19) United States
(12) Patent Application Publication
(10) Pub. No.: US 2019/0094191 A1
(43) Pub. Date: Mar. 28, 2019

(54) METHOD AND SYSTEM FOR MEASURING CONTENT OF DISSOLVED ORGANIC HALOGENS IN WATER

(71) Applicant: HARBIN INSTITUTE OF TECHNOLOGY SHENZHEN GRADUATE SCHOOL, Shenzhen (CN)

(72) Inventors: Baiyang CHEN, Shenzhen (CN); Yinan BU, Shenzhen (CN)

(73) Assignee: HARBIN INSTITUTE OF TECHNOLOGY SHENZHEN GRADUATE SCHOOL, Shenzhen (CN)

(21) Appl. No.: 16/199,325

(22) Filed: Nov. 26, 2018

Related U.S. Application Data
(63) Continuation of application No. PCT/CN2017/000244, filed on Mar. 21, 2017.

(30) Foreign Application Priority Data
May 26, 2016 (CN) 201610362531.4

(51) Int. Cl. G01N 30/96; G01N 1/34; G01N 33/18; G01N 1/40

(52) U.S. Cl. CPC G01N 30/96; G01N 1/34; G01N 2001/4038; G01N 1/4005; G01N 2001/4011; G01N 33/1826

(57) ABSTRACT

A method and system for measuring the content of dissolved organic halide in water. The method includes separating dissolved inorganic halides and dissolved organic halides in water using an electrodialysis technique (S1), which may completely separate the dissolved organic halides and the dissolved inorganic halides, avoid the use of activated carbon and adsorption columns, simplify steps and improve efficiency. The method converts the separated dissolved organic halides into dissolved inorganic halides using a photocatalysis technique (S2), which does not need a special high-temperature combustion device, active carbon and catalysts, and is simple and easy to be performed at room temperature in more environmentally friendly mode. The method also includes analyzing the converted dissolved inorganic halides using a device with an ion analysis function (S3) to analyze and measure a total content index of the dissolved organic halides and an individual content index of each of the dissolved organic halides (fluorine, chlorine, bromine and iodine) in water to be measured.

### CROSS-REFERENCE TO RELATED APPLICATIONS

This application is a continuation of International Application No. PCT/CN2017/000244, filed on Mar. 21, 2017, which claims priority to Chinese Application No. 201610362531.4, filed on May 26, 2016. The above-referenced applications are incorporated herein by reference in their entirety.

### TECHNICAL FIELD

The present disclosure relates to the field of measurement of halogenated substances, and more particularly, relates to a method and system for measuring the content of dissolved organic halide in water.

### BACKGROUND

Dissolved organic halide (DOH) is an indicator of the total content of halogenated substances in water and beverages. Because dissolved inorganic halide (DIH) and DOH coexist in water and DIH is the dominant ingredient, DOH cannot be directly measured using prior art technologies. Only by removing the dominant ingredient DIH before DOH measurement, and converting DOH into DIH, the DOH in water sample may be measured. Currently, a common DOH measurement method may be divided into three steps including separating DOH and DIH using activated carbon, converting DOH to DIH by combustion technique, measuring the converted DIH after combustion.

The current measurement method uses activated carbons to intercept DOH by exploiting the difference between DOH and DIH in the adsorption performance on activated carbon. However, during the interception process, part of hydrophilic DOH may be lost with the solution, thus reducing the recovery of DOH and increasing the measurement error of DOH. Moreover, due to the reduction potential of activated carbon, organic chlorine and organic bromine in DOH may be reduced and dehalogenated, further reducing the recovery of DOH and increasing the measurement error of DOH. In addition, the method employs a special adsorption column made of activated carbon. The adsorption column is expensive, non-reuseable, and is not environmentally friendly, which needs to be replaced after each use, thereby increasing the cost, difficulty, and complexity of each operation such as replacing adsorption column.

In the second step of the current measurement method, activated carbon and DOH adsorbed by activated carbon are to be converted into inorganic carbon and DIH, respectively, by high-temperature combustion which needs a high temperature and high performance combustion equipment. The manufacture and operation of combustion equipment are also inconvenient and not environmentally friendly. In addition, in order to increase the conversion rate of DOH, it is necessary to add a catalyst (e.g., a noble metal catalyst-platinum). However, since halogen ions can quench the activity of catalyst, it is infeasible to use a catalyst to convert DOH at a relatively low temperature (e.g., 680° C.). The combustion temperature need to be raised above 950° C., which demands high performance material for the combustion equipment. In addition, the cooling process after high-temperature combustion causes condensation of halides. For example, trifluoroacetic acid is decomposed into hydrogen fluoride after high temperature combustion, which may react with water in air to form hydrofluoric acid during the cooling process, reducing the recovery rate of trifluoroacetic acid and further increasing the measurement error of DOH.

In the third step of the current measurement method, a microcoulometric technique and an ion chromatography (IC) technique may be used. Using the microcoulometric technique, the total amount of halide may be measured based on the current change caused by precipitates generated by a reaction between halide ions and silver ions. However, as silver fluoride does not form a precipitate, it is infeasible to measure fluorine ions in organic halide, which increases the measurement error of DIH and also the total measurement error of DOH. Moreover, since chlorine ions, bromine ions and iodine ions all have identical precipitation effect, it is infeasible to distinguish the contents of chlorine ions, bromine ions, and iodine ions in DIH. Using the IC technique, the total amount of halide may be measured based on the conductance change of effluents including inorganic ions detected continuously by an IC conductivity detector. The inorganic ions are separated by using an anion or cation ion exchange column with certain low-exchange capacity and by using a strong electrolyte as a flow phase. The IC technique solves the problem that fluorine ions cannot be measured using the microcoulometric technique and the content of chlorine and bromine cannot be distinguished. However, an IC conductivity detector have a lower response signal to iodine ions, while the content of iodide in drinking water is as low as a few micrograms per liter or even nanograms per liter. Therefore, the technique using the IC conductivity detector cannot be used for the detection of trace iodine ions, and cannot effectively and accurately detect the iodine content in drinking water.

### SUMMARY

Current methods for measuring a dissolved organic halide have problems that the measurement error is large, the test steps are cumbersome, the test environment is demanding, not environmentally friendly, and the test efficiency is low. In order to solve the above problems, the present disclosure provides a simple and environmentally friendly testing technique with high test efficiency, small measurement error, high measurement accuracy, and low requirements for testing environment.

The method and system disclosed herein may measure dissolved organic halide at room temperature.

The technique of the present disclosure is as follows.

The present disclosure provides a method for measuring content of a dissolved organic halide in water, including:

separating a dissolved inorganic halide and a dissolved organic halide in a water sample to be measured using an electrodialysis technique;

converting the separated dissolved organic halide into a dissolved inorganic halide using a photocatalytic technique;

measuring a content of the dissolved organic halide in the water sample to be measured by analyzing the converted dissolved inorganic halide using an ion analysis instrument.

In some embodiments, the separating the dissolved inorganic halide and the dissolved organic halide in the water sample to be measured using an electrodialysis technique may include:

separating the dissolved inorganic halide and the dissolved organic halide in the water sample to be measured in an electrodialyser using the electrodialysis technique.

In some embodiments, the separating the dissolved inorganic halide and the dissolved organic halide in the water sample to be measured in an electrodialyser using the electrodialysis technique may include:

providing the electrodialyser including a first concentration chamber, a dilution chamber, and a second concentration chamber arranged in order, the dilution chamber being connected with the first concentration chamber and the second concentration chamber, respectively, via one or more exchange membranes;

injecting an inorganic electrolyte solution excluding halogens into the first concentration chamber and the second concentration chamber;

injecting the water sample to be measured into the dilution chamber;

applying a voltage to a first electrode and a second electrode to generate an electric field that drives inorganic halogen ions in the dilution chamber to flow into the first concentration chamber or the second concentration chamber through the exchange membranes; and

taking out the water sample including the dissolved organic halide from the dilution chamber to measure the content of the dissolved organic halides from the water sample to be measured. In some embodiments, the first electrode may be configured in the first concentration chamber. The second electrode may be configured in the second concentration chamber. polarity of an exchange membrane connecting the first concentration chamber and the dilution chamber may be the same as the second electrode. A polarity of an exchange membrane connecting the second concentration chamber and the dilution chamber may be the same as the first electrode.

In some embodiments, the converting the separated dissolved organic halide into a dissolved Inorganic halide using a photocatalytic technique may include:

converting the dissolved organic halide in the water sample to the dissolved inorganic halide by irradiating with ultraviolet (UV) light the water sample obtained from the dilution chamber.

According to another aspect of the present disclosure, a system for measurement of dissolved organic halide content in water may be provided. The system may use a method for measurement of dissolved organic halide content in water as described above. The system may include:

a separation device configured to separate a dissolved inorganic halide and a dissolved organic halide in a water sample to be measured using an electrodialysis technique;

a photocatalytic device configured to convert the separated dissolved organic halide into a dissolved Inorganic halide using a photocatalytic technique; and

an ion analysis device configured to analyze and measure a content of the dissolved organic halide in the water sample to be measured.

In some embodiments, the separation device may include an electrodialyser. The electrodialyser may include a first concentration chamber, a dilution chamber, and a second concentration chamber arranged in order. The dilution chamber may be connected with the first concentration chamber and the second concentration chamber, respectively, via one or more exchange membranes. The first concentration chamber may be configured with a first electrode. The second concentration chamber may be configured with a second electrode. The first concentration chamber and the second concentration chamber may be configured to receive an injected inorganic electrolyte solution excluding halogens. The dilution chamber may be configured to receive the injected water sample to be measured. The first electrode and the second electrode may be configured to generate an electric field by applying a voltage on the first electrode and the second electrode. The electric field may drive inorganic halogen ions in the dilution chamber to flow into the first concentration chamber or the second concentration chamber through the exchange membranes to separate the dissolved inorganic halide and the dissolved organic halide in the water sample to be measured.

In some embodiments, the first concentration chamber may include a body, and a water outlet and a water inlet configured on the body of the first concentration chamber. The second concentration chamber may include a body, and a water outlet and a water inlet configured on the body of the second concentration chamber. The water outlet and the water inlet of each of the first concentration chamber and the second concentration chamber may be configured to maintain a halogen ion concentration in the first concentration chamber and the second concentration chamber by coordinately adjusting a flow rate through the water outlet and a flow rate through the water inlet of each of the first concentration chamber and the second concentration chamber.

In some embodiments, the separation device may further include an electrolyte solution adder connected to the water inlet of each of the first concentration chamber and the second concentration chamber. The electrolyte solution adder may be configured to add and/or inject an inorganic electrolyte solution to the first concentration chamber and the second concentration chamber. The electrolyte solution adder may control a flow rate of the water outlet and the water inlet to maintain a halogen ion concentration in the first concentration chamber and the second concentration chamber by coordinately adjusting the flow rate through the water outlet and the flow rate through the water inlet of each of the first concentration chamber and the second concentration chamber.

In some embodiments, the exchange membranes may be configured to separate the dissolved organic halide and the dissolved inorganic halide. The exchange membranes may include an ion exchange membrane, a bipolar membrane, a reverse osmosis (RO) membrane, a nanofiltration membrane, or a dialysis membrane.

In some embodiments, the photocatalytic device may include an ultraviolet irradiation device configured to convert the separated dissolved organic halide into the dissolved inorganic halide using the photocatalytic technique. The ultraviolet irradiation device may irradiate the water sample obtained from the dilution chamber to convert the dissolved organic halide in the water sample into the dissolved inorganic halide. The ultraviolet lamp may include a low pressure mercury lamp, a medium pressure UV lamp, a high pressure mercury lamp, an amalgam UV lamp, an excimer excitation UV lamp, an xenon lamp, or a halide lamp.

The benefits of the present disclosure include: the method and system for measuring the total content of dissolved organic halide in water may separate dissolved inorganic halide and dissolved organic halide in water to be measured using an electrodialysis technique, which may completely separate dissolved organic halide and dissolved inorganic halide ions, less cumbersome, and be environmentally friendly. The method does not involve replacing testing supplies every time, which may simplify testing operations and improve test efficiency. The method may convert the separated dissolved organic halide into dissolved inorganic halide using a photocatalytic technique which does not involve special high-temperature combustion equipment and may be easy to perform. Using the photocatalytic technique, the method may be performed at room temperature, and does not need to heat and cool a sample, which may avoid the stringent environmental requirements of employing special high-temperature combustion equipment for conversion, and improve the test efficiency. It does not need to burn activated carbon and add catalyst, which is more environmentally friendly. The method may analyze the converted dissolved inorganic halide using an instrument with an ion analysis ability to analyze the content of dissolved organic halide in a water sample to be measured, which may achieve accurate measurement of the total dissolved organic halide content in water, and the individual contents of fluorine, chlorine, bromine, and iodine in the dissolved organic halide.

### BRIEF DESCRIPTION OF THE DRAWINGS

FIG. 1a is a flowchart illustrating a process for measuring the content of dissolved organic halide in water according to some embodiments of the present disclosure;

FIG. 1b is a flowchart illustrating another process for measuring the content of dissolved organic halide in water according to some embodiments of the present disclosure;

FIG. 2 is a flowchart illustrating another process for measuring the content of dissolved organic halide in water according to some embodiments of the present disclosure;

FIG. 3 is a diagram illustrating an electrodialyser for measuring the content of dissolved organic halide in water according to some embodiments of the present disclosure;

FIGS. 4a and 4b are flowcharts illustrating another process for measuring the content of dissolved organic halide in water according to some embodiments of the present disclosure;

FIG. 5 is a flowchart illustrating another process for measuring the content of dissolved organic halide in water according to some embodiments of the present disclosure;

FIG. 6 is a flowchart illustrating another process for measuring the content of dissolved organic halide in water according to some embodiments of the present disclosure; and

FIG. 7 is a flowchart illustrating another process for measuring the content of dissolved organic halide in water according to some embodiments of the present disclosure.

### DETAILED DESCRIPTION

The embodiments set forth below represent the necessary information for practicing the present disclosure by those skilled in the art, and show the best way to practice the present disclosure. The following descriptions of the present disclosure will be understood by those skilled in the art and will recognize the application of these concepts not specifically set forth herein. It should be understood that these concepts and applications fall within the scope of the disclosure and the appended claims. The present disclosure is further described below in conjunction with the examples.

The technical proposal of the present disclosure is as follows.

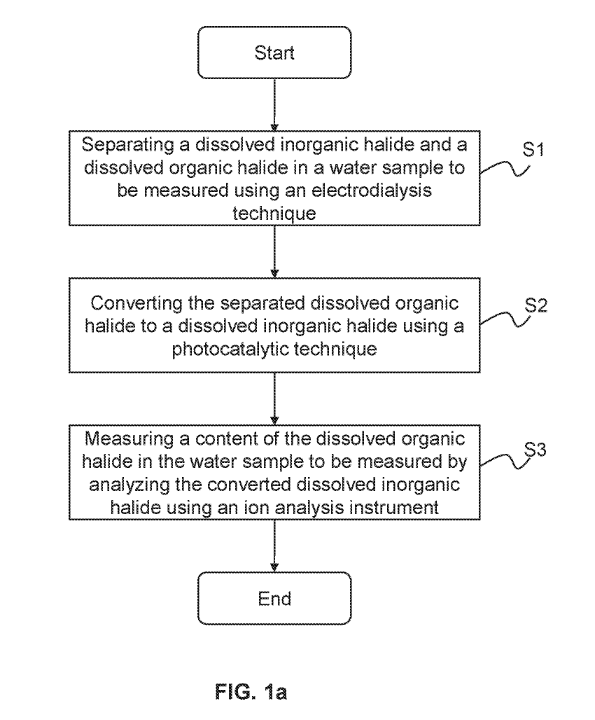

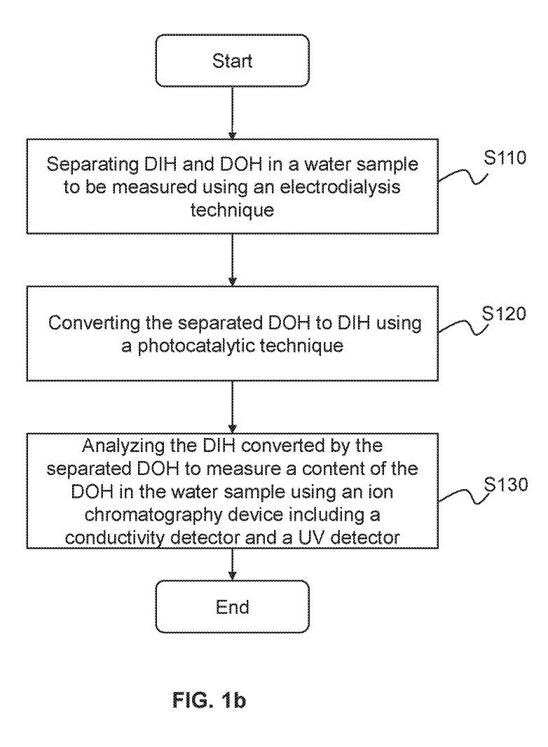

FIG. 1a is a flowchart illustrating a process for measuring content of dissolved organic halide in water according to some embodiments of the present disclosure. The process for measuring content of dissolved organic halide in water may include:

S1, separating a dissolved inorganic halide and a dissolved organic halide in a water sample to be measured using an electrodialysis technique;

S2, converting the separated dissolved organic halide to a dissolved inorganic halide using a photocatalytic technique;

S3, measuring a content of the dissolved organic halide in the water sample to be measured by analyzing the converted dissolved inorganic halide using an ion analysis instrument.

In some embodiments, the separating the dissolved inorganic halide and the dissolved organic halide in the water sample to be measured using an electrodialysis technique may include:

separating the dissolved inorganic halide and the dissolved organic halide in the water sample to be measured in an electrodialyser using the electrodialysis technique.

In some embodiments, the separating the dissolved inorganic halide and the dissolved organic halide in the water sample to be measured in an electrodialyser using the electrodialysis technique may include:

providing the electrodialyser including a first concentration chamber, a dilution chamber, and a second concentration chamber arranged in order, the dilution chamber being connected with the first concentration chamber and the second concentration chamber, respectively, via one or more exchange membranes;

injecting an inorganic electrolyte solution excluding halogens into the first concentration chamber and the second concentration chamber;

injecting the water sample to be measured into the dilution chamber;

applying a voltage or current to a first electrode and a second electrode to generate an electric field driving inorganic halogen ions in the dilution chamber to flow into the first concentration chamber or the second concentration chamber through the exchange membranes; and

taking out the water sample including the dissolved organic halide from the dilution chamber to measure the content of the dissolved organic halides from the water sample to be measured. In some embodiments, the first electrode may be configured in the first concentration chamber. The second electrode may be configured in the second concentration chamber. A polarity of an exchange membrane connecting the first concentration chamber and the dilution chamber may be the same as the second electrode. A polarity of an exchange membrane connecting the second concentration chamber and the dilution chamber may be the same as the first electrode.

In some embodiments, using the electrodialysis technique, the dissolved inorganic halide in the water sample in the dilution chamber is dissociated and exists in a variety of forms. The exchange membrane between a concentration chamber (e.g., the first concentration chamber, the second concentration chamber) and the dilution chamber may be semi-permeable. The inorganic halogens in the dilution chamber may pass through one or more exchange membranes and flow into the first concentration chamber and/or the second concentration chamber driven by the electric field, but not in the opposite direction, i.e., from the first concentration chamber and/or the second concentration chamber into the dilution chamber under the electric field. Therefore in the dilution chamber, dissolved inorganic halide is removed from the water sample, and the content of the dissolved inorganic halide is reduced. As a consequence, the dissolved organic halide becomes the dominant ingredient in the water sample remaining in the dilution chamber; the resultant water sample in the dilution chamber may be used for measuring the content of dissolved organic halide.

In some embodiments, the converting the separated dissolved organic halide into a dissolved inorganic halide using a photocatalytic technique may include: converting the dissolved organic halide in the water sample to the dissolved inorganic halide by irradiating with ultraviolet (UV) light the water sample obtained from the dilution chamber.

In some embodiments, the separated dissolved organic halide may be converted into dissolved inorganic halide using one or more non-combustion technique not employing combustion of the dissolved organic halide. For example, other than UV irradiation as described, the separated dissolved organic halide may be converted into dissolved inorganic halide using an alkaline oxidation technique, an alkaline reduction technique, a circumfluence technique, a laser irradiation technique, a neutron activation technique, or the like, or a combination thereof. The separated dissolved organic halide may be converted to dissolved inorganic halide at a temperature lower than an ignition point of the separated dissolved organic halide, for example, at room temperature.

According to another aspect of the present disclosure, a system for measurement of dissolved organic halide content in water may be provided. The system may use a method for measurement of dissolved organic halide content in water as described above. The system may include:

a separation device configured to separate a dissolved inorganic halide and a dissolved organic halide in a water sample to be measured using an electrodialysis technique;

a photocatalytic device configured to convert the separated dissolved organic halide into a dissolved Inorganic halide using a photocatalytic technique; and

an ion analysis device configured to analyze and measure a content of the dissolved organic halide in the water sample to be measured.

In some embodiments, the separation device may include an electrodialyser. The electrodialyser may include a first concentration chamber, a dilution chamber, and a second concentration chamber arranged in order. The dilution chamber may be connected with the first concentration chamber and the second concentration chamber, respectively, via one or more exchange membranes. The first concentration chamber may be configured with a first electrode. The second concentration chamber may be configured with a second electrode. The first concentration chamber and the second concentration chamber may be configured to receive an injected inorganic electrolyte solution without halogens. The dilution chamber may be configured to receive the injected water sample to be measured. The first electrode and the second electrode may be configured to generate an electric field by applying a voltage or a current on the first electrode and the second electrode. The electric field may drive inorganic halogen ions in the dilution chamber to flow into the first concentration chamber or the second concentration chamber through the exchange membranes to separate the dissolved inorganic halide and the dissolved organic halide in the water sample to be measured.

In some embodiments, the first concentration chamber may include a body, a water outlet and a water inlet configured on the body of the first concentration chamber. The second concentration chamber may include a body, a water outlet and a water inlet configured on the body of the second concentration chamber. The water outlet and the water inlet of each of the first concentration chamber and the second concentration chamber may be configured to maintain a low halogen ion concentration in the first concentration chamber and the second concentration chamber by coordinately adjusting the flow rate through the water outlet and the flow rate through the water inlet of each of the first concentration chamber and the second concentration chamber.

In some embodiments, using the electrodialysis technique, as the electrodialysis process progresses, dissolved inorganic halide in the dilution chamber may flow into and concentrate in the first concentration chamber and/or the second concentration chamber. The concentration of halogens in the first concentration chamber and/or the second concentration chamber may accumulate such that may prevent more halogens in the dilution chamber from flowing into the first concentration chamber and/or the second concentration chamber. The solution with halogen accumulation in the first concentration chamber and the second concentration chamber may be discharged from the first concentration chamber and/or the second concentration chamber via the water outlet. The inorganic electrolyte solution without halogens may be injected into the first concentration chamber and/or the second concentration chamber via the water inlet. As used herein, the content of halogen in an inorganic electrolyte solution without halogens may be lower than 20 micrograms per liter, or lower than 15 micrograms per liter, or lower than 10 micrograms per liter. Thereby the halogen ion concentration in the first concentration chamber and the second concentration chamber may be maintained according to or not exceeding a concentration threshold by coordinately adjusting the flow rate through the water outlet and the flow rate through the water inlet of the first concentration chamber and/or the second concentration chamber.

In some embodiments, the separation device may further include an electrolyte solution adder connected to the water inlet of each of the first concentration chamber and the second concentration chamber. The electrolyte solution adder may be configured to add and/or inject an inorganic electrolyte solution to the first concentration chamber and the second concentration chamber. The electrolyte solution adder may control the flow rate of the water outlet and the water inlet to maintain a halogen ion concentration in the first concentration chamber and the second concentration chamber by coordinately adjusting the flow rate through the water outlet and the flow rate through the water inlet of each of the first concentration chamber and the second concentration chamber.

In some embodiments, the exchange membranes may be configured to separate the dissolved organic halide and the dissolved inorganic halide. The exchange membranes may include an ion exchange membrane, a bipolar membrane, a reverse osmosis (RO) membrane, a nanofiltration membrane, or a dialysis membrane.

In some embodiments, the photocatalytic device may include an ultraviolet irradiation device configured to convert the separated dissolved organic halide into the dissolved inorganic halide using the photocatalytic technique. The ultraviolet irradiation device may irradiate the water sample obtained from the dilution chamber to convert the dissolved organic halide in the water sample into the dissolved inorganic halide. The ultraviolet lamp may include a low pressure mercury lamp, a medium pressure UV lamp, a high pressure mercury lamp, an amalgam UV lamp, an excimer excitation UV lamp, an xenon lamp, or a halide lamp.

The benefits of the present disclosure include: the method and system for measuring the total content of dissolved organic halide in water may separate dissolved inorganic halide and dissolved organic halide in water to be measured using an electrodialysis technique, which may completely separate dissolved organic halide ions and dissolved inorganic halide ions, save energy and resources, be environmentally friendly. The method does not involve replacing testing supplies every time, which may simplify testing operations and improve test efficiency. The method may convert the separated dissolved organic halide into dissolved inorganic halide using a photocatalytic technique which does not involve special high-temperature combustion equipment and may be easy to perform. Using the photocatalytic technique, the method may be performed at room temperature, and does not need to heat and take time to cool a sample, which may avoid the stringent environmental requirements of employing special high-temperature combustion equipment for conversion, and improve the test efficiency.

It does not need to burn activated carbon and add a catalyst, which is more environmentally friendly. The method may analyze the converted dissolved inorganic halide using an instrument with an ion analysis function to analyze the content of dissolved organic halide in a water sample to be measured, which may achieve accurate measurement of the total dissolved organic halide content in water, and the individual contents of fluorine, chlorine, bromine, and iodine in the total dissolved organic halide. FIG. 1b is a flowchart illustrating another process for measuring content of dissolved organic halide in water according to some embodiments of the present disclosure. The process for measuring content of dissolved organic halide in water may include:

S110, separating DIH and DOH in a water sample to be measured using an electrodialysis technique;

S120, converting the separated DOH into DIH using a photocatalytic technique;

S130, analyzing the DIH converted by the separated DOH to measure a content of the DOH in the water sample using an ion chromatography device including a conductivity detector and a UV detector. As fluorine, chlorine, and bromine have strong response signals to the conductivity detector, and bromine and iodine have strong response signals to the ultraviolet detector, measurements of four halide ions (i.e., fluorine, chlorine, bromine, and iodine) of low concentration may be simultaneously achieved using the dual detectors.

In S110, the dissolved inorganic halide and dissolved organic halide in water to be measured may be separated using an electrodialysis technique, which may completely separate dissolved organic halide ions and dissolved inorganic halide ions, save energy and resources, be environmentally friendly. The method does not involve replacing testing supplies every time, which may simplify testing operations and improve test efficiency. In S120, the separated dissolved organic halide may be converted into dissolved inorganic halide using a photocatalytic technique which does not involve special high-temperature combustion equipment and may be easy to perform. Using the photocatalytic technique, the method may be performed at room temperature, and does not need to heat and take time to cool a sample, which may avoid the stringent environmental requirements of employing special high-temperature combustion equipment for conversion, and improve the test efficiency. It does not need to burn activated carbon and add a catalyst, which is more environmentally friendly. In S130, the converted dissolved inorganic halide may be analyzed using an ion chromatography device including a conductivity detector and a UV detector to measure the content of the dissolved organic halide in the water to be measured.

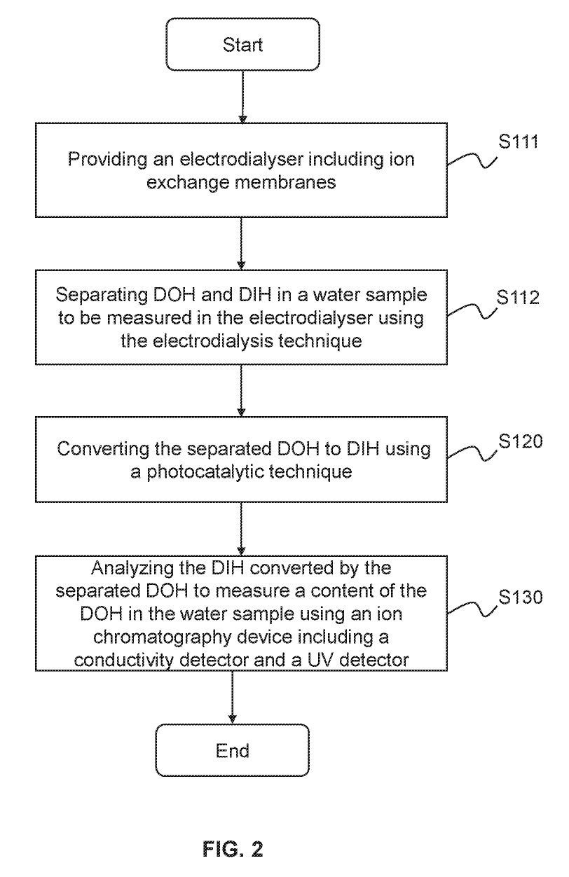

FIG. 2 is a flowchart illustrating another process for measuring content of dissolved organic halide in water according to some embodiments of the present disclosure. Step S110 as described in FIG. 1b may be performed according to FIG. 2. Step S110 may include:

S111, providing an electrodialyser including ion exchange membranes; and

S112, separating DOH and DIH in a water sample to be measured in the electrodialyser using the electrodialysis technique.

According to the present disclosure, the dissolved inorganic halide and dissolved organic halide may be separated using ion exchange membranes, which may completely separate dissolved organic halide ions and dissolved inorganic halide ions, save resources, be environmentally friendly. The method does not involve replacing testing supplies every time, which may simplify testing operations and improve test efficiency.

In some embodiments, the ion exchange membranes may be configured to separate the dissolved organic halide and the dissolved inorganic halide. The ion exchange membranes may include an ion exchange membrane, a bipolar membrane, a reverse osmosis (RO) membrane, a nanofiltration membrane, or a dialysis membrane. The specific implementation of the exchange membranes is not limited by the embodiment.

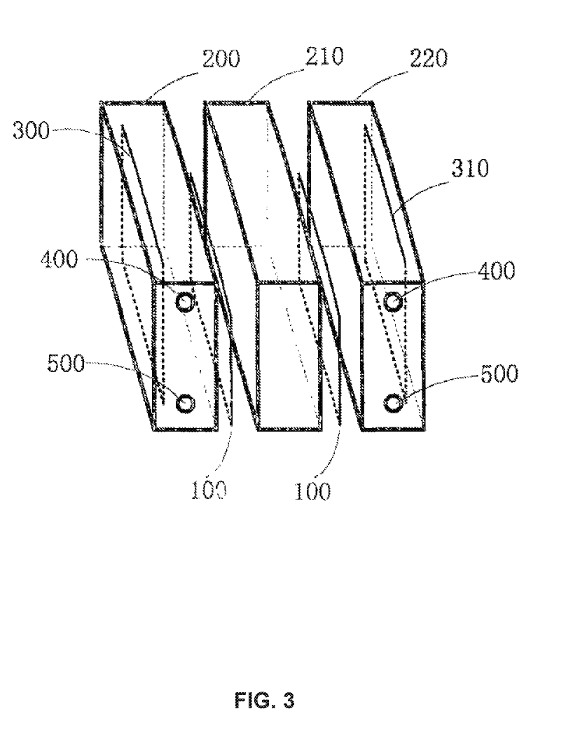

FIG. 3 is a diagram illustrating an electrodialyser for measuring content of dissolved organic halide in water according to some embodiments of the present disclosure. The electrodialyser as described in step S111 may be described as follows.

The electrodialyser may include a first concentration chamber 200, a dilution chamber 210, and a second concentration chamber 220 arranged in order and connected by exchange membranes 100. The first concentration chamber 200 may be configured with a first electrode 300. The second concentration chamber 220 may be configured with a second electrode 310. The polarity of an exchange membrane 100 connecting the first concentration chamber 200 and the dilution chamber 210 may be the same as the second electrode 310. The polarity of an exchange membrane 100 connecting the second concentration chamber 220 and the dilution chamber 210 may be the same as the first electrode 300.

Specifically, in step S112, the separating of DOH and DIH in a water sample to be measured in the electrodialyser using the electrodialysis technique may include:

injecting an inorganic electrolyte solution excluding halogens into the first concentration chamber 200 and the second concentration chamber 220, and injecting a water sample to be measured into the dilution chamber 210;

applying a voltage or a current to the first electrode 300 and the second electrode 310 to generate an electric field driving inorganic halogen ions in the dilution chamber 210 to flow into the first concentration chamber 200 or the second concentration chamber 220 through the exchange membranes 100;

taking out the water sample including the DOH from the dilution chamber 210, with the DIH and the DOH in the water sample to be measured having been separated.

Specifically, the applying a voltage or a current to the first electrode 300 and the second electrode 310 to generate an electric field driving inorganic halogen ions in the dilution chamber 210 to flow into the first concentration chamber 200 or the second concentration chamber 220 through the exchange membranes 100 may further include:

applying a direct voltage or a current not exceeding 30V to the first electrode 300 and the second electrode 310 for 20 minutes to 100 minutes. The direct voltage or a current may generate an electric field driving inorganic halogen ions in the dilution chamber 210 to flow into the first concentration chamber 200 or the second concentration chamber 220 through the exchange membranes 100.

In some embodiments, the electrolyte solution may include a sodium hydrogencarbonate solution having a concentration of 0.01 mol/L.

In some embodiments, before injecting an inorganic electrolyte solution without halogens into the first concentration chamber 200 and the second concentration chamber 220, and injecting a water sample to be measured into the dilution chamber 210, each of the first concentration chamber 200 and the second concentration chamber 220 may be configured with a water outlet 400 and a water inlet 500.

In some embodiments, after applying a voltage or a current to the first electrode 300 and the second electrode 310 to generate an electric field driving inorganic halogen ions in the dilution chamber 210 to flow into the first concentration chamber 200 or the second concentration chamber 220 through the exchange membranes 100, the method may further include:

injecting the inorganic electrolyte solution without halogens into the first concentration chamber 200 and the second concentration chamber 220 via the water inlet 500 according to a constant or specific flow rate. A halogen ion concentration in the first concentration chamber 200 and the second concentration chamber 220 may be maintained by coordinately adjusting the flow rate through the water outlet 400 and the flow rate through the water inlet 500 of each of the first concentration chamber and the second concentration chamber.

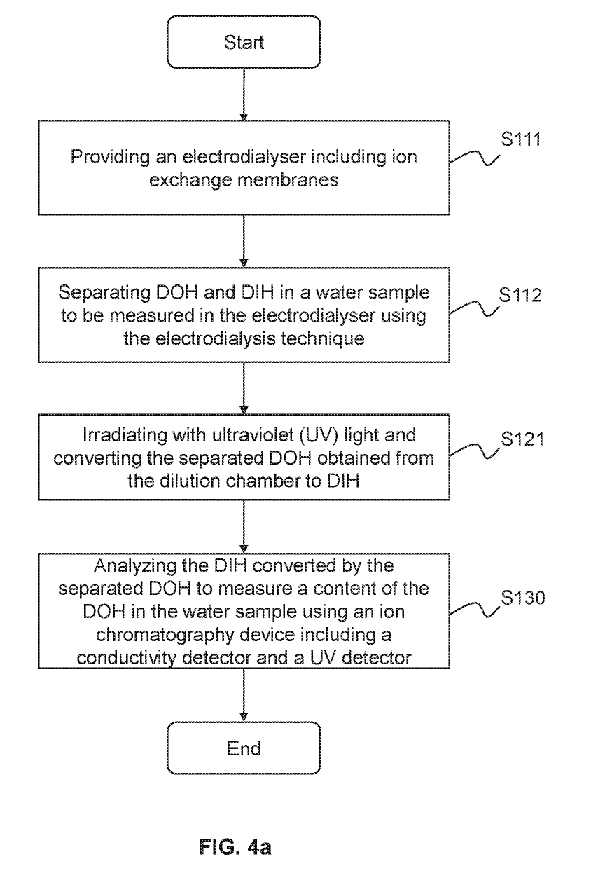

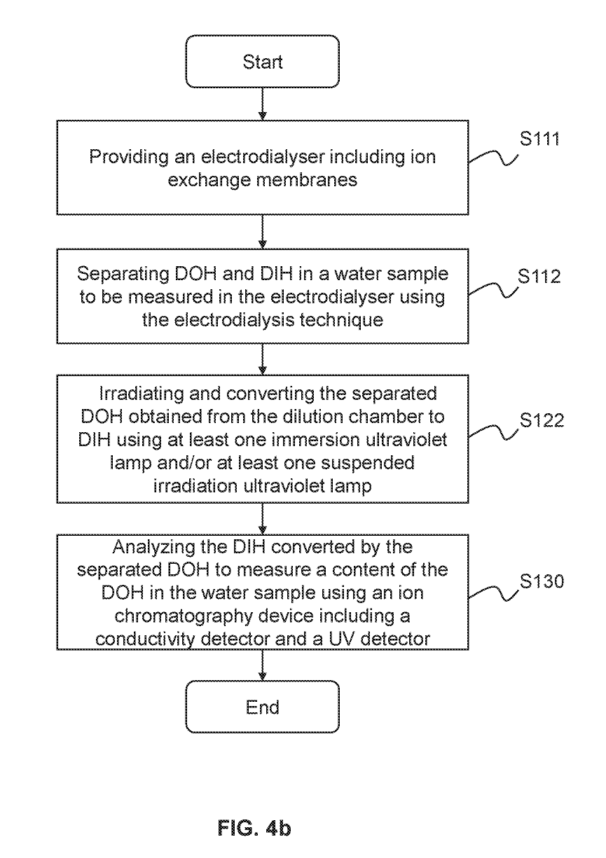

FIGS. 4a and 4b provide flowcharts illustrating another process for measuring the content of dissolved organic halide in water according to some embodiments of the present disclosure. According to operations illustrated in FIG. 2, operation S120 may be performed according to FIG. 4a. Operation S120 may include:

S121, irradiating with ultraviolet (UV) light and converting the separated DOH obtained from the dilution chamber to DIH.

Further, as shown in FIG. 4b, operation S121 as illustrated in FIG. 4a may further include:

S122, irradiating and converting the separated DOH obtained from the dilution chamber into DIH using at least one immersion ultraviolet lamp and/or at least one suspended irradiation ultraviolet lamp. The at least one immersion ultraviolet lamp or at least one suspended irradiation ultraviolet lamp may include a low pressure mercury lamp, a medium pressure UV lamp, a high pressure mercury lamp, an amalgam UV lamp, an excimer excitation UV lamp, an xenon lamp, or a halide lamp. The UV irradiation time may be less than or equal to 120 minutes, and the UV irradiation dosage may be less than or equal to 90,000 J/L.

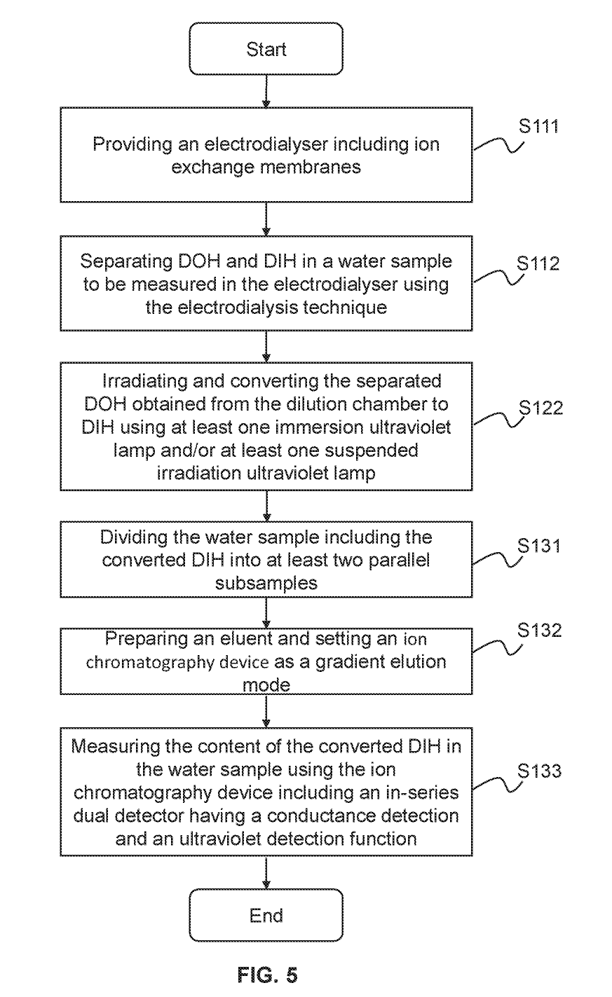

FIG. 5 is a flowchart illustrating another process for measuring the content of dissolved organic halide in water according to some embodiments of the present disclosure. According to operations illustrated in FIG. 4, operation S130 as described in FIG. 1b may be performed according to FIG. 5. Operation S130 may include:

S131, dividing the water sample including the converted DIH into at least two parallel subsamples, and preparing an eluent;

S132, setting an ion chromatography device as a gradient elution mode;

S133, measuring the content of the converted DIH in the water sample using the ion chromatography device including in-series dual detectors having a conductance detection and an ultraviolet detection function.

When step S132 and step S133 is performed, the ion chromatography device may be set to be ran at a gradient elution mode, and then it starts to work. An eluent may enter the ion chromatography device, and the at least two parallel subsamples may be injected into the ion chromatography device to check the consistency of chromatographic columns. The chromatographic separation of the at least two parallel subsamples may be carried out using the gradient elution mode. Ions to be tested in the eluent flowed from the ion chromatography device may be obtained. And, then operation S133 may be performed. As used herein, two parallel subsamples refer to that the two parallel subsamples having a same volume are tested using the ion chromatography device at a same operation condition, such as at a same temperature, at a same power, at a same pressure, at a same eluent, at a same elution mode, etc.

In some embodiments, the eluent is generated and provided by an eluent producing component of the ion chromatography device according to the gradient elution mode. Using the gradient elution mode, during an elution process of samples, the property of the eluent (e.g., a concentration ratio of solutions in the eluent, a pH value, a polarity, etc.) may be adjusted by the eluent producing component according to the gradient elution mode, which can help separate different halogens in the converted DIH more efficiency. Each of the at least two parallel subsamples may be injected into and eluted out by the ion chromatography device using the eluent. Different types of halogens (e.g., fluorine, chlorine, bromine, iodine, etc.) may be separated in the ion chromatography device in the elution process. The content of the produced DIH in the water sample may be determined based on the total content of different types of halogens in the water sample including the converted DIH.

In some embodiments, the method may further include measuring the content of each of halogens in the converted DIH using the ion chromatography device including in-series dual detectors having a conductance detection and an ultraviolet detection function. For example, the ion chromatography device may include a conductivity detector and a UV detector. The conductivity detector may be used to detect fluorine, chlorine, and bromine having strong response signals to the conductivity detector. The UV detector may be used to detect bromine and iodine having strong response signals to the ultraviolet detector.

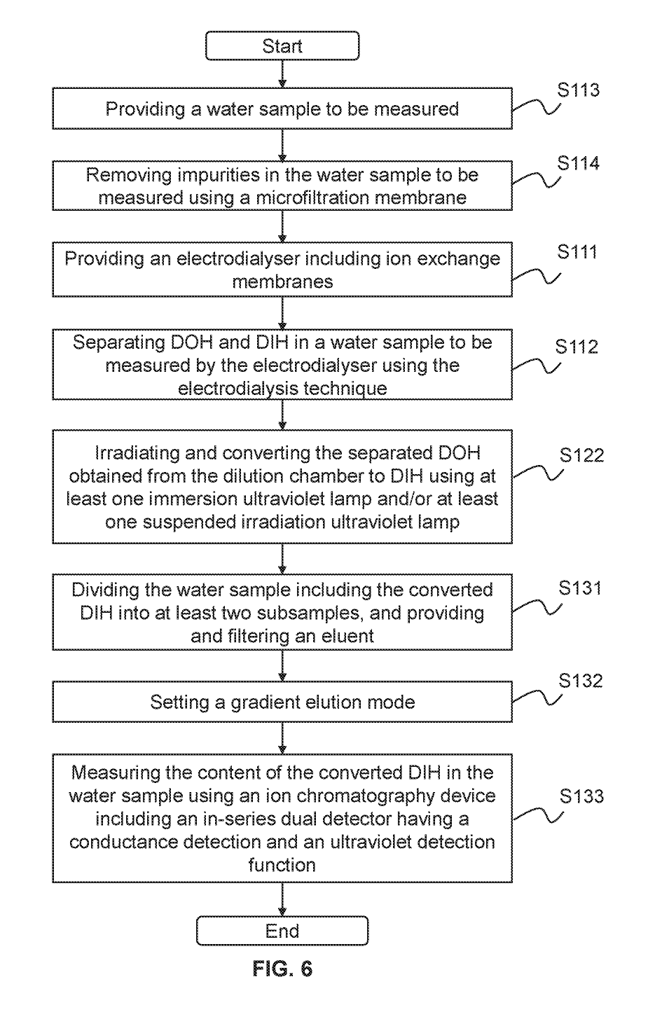

FIG. 6 is a flowchart illustrating another process for measuring the content of dissolved organic halide in water according to some embodiments of the present disclosure. Before operation S111 is performed as described in FIG. 2 based on operations as illustrated in FIG. 5, the method may further include:

S113, providing a water sample to be measured;

S114, removing impurities in the water sample to be measured using a microfiltration membrane.

Specifically, a pore diameter of the microfiltration membrane may be not less than 0.22 micrometers and not more than 0.7 micrometers.

The chromatographic separation of the at least two subsample may be carried out using the gradient elution mode. Ions to be tested in the eluent flowed from the ion chromatography device may be obtained.

The concentration of Na₂CO₃ in the ion chromatography device may be 2.2 mmol/I and the concentration of NaHCO₃ in the ion chromatography device may be 1.5 mmol/I.

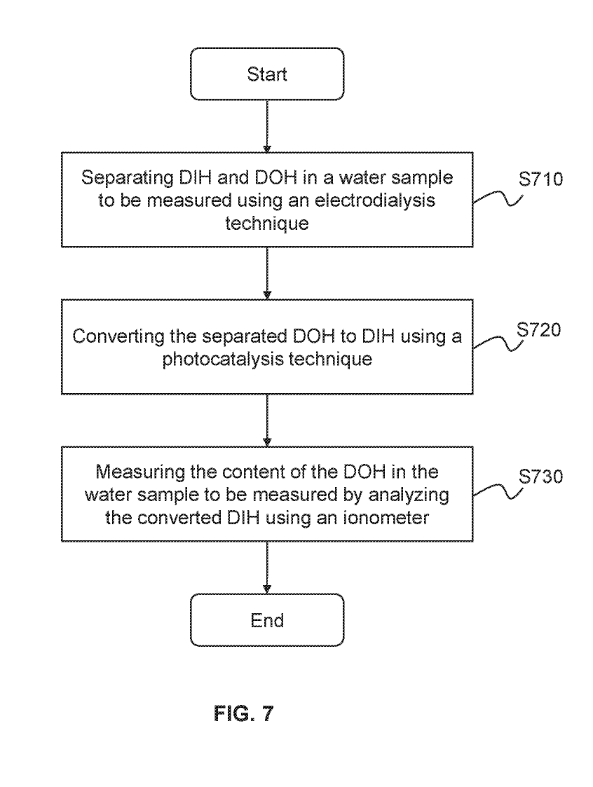

FIG. 7 is a flowchart illustrating another process for measuring content of dissolved organic halide in water according to some embodiments of the present disclosure. The process for measuring content of dissolved organic halide in water may include:

S710, separating DIH and DOH in a water sample to be measured using an electrodialysis technique;

S720, converting the separated DOH to DIH using a photocatalysis technique;

S730, measuring the content of the DOH in the water sample to be measured by analyzing the converted DIH using an ionometer.

In S710, the dissolved inorganic halide and dissolved organic halide in water to be measured may be separated using an electrodialysis technique. Using the electrodialysis technique, the DIH having a smaller diameter may be easy to penetrate an electrodialyser and the DOH having a larger diameter may be intercepted by the electrodialyser under restriction of a bore diameter of the electrodialyser, which may completely separate dissolved organic halide ions and dissolved inorganic halide ions, save resources, be environmentally friendly, but not involve replacing testing supplies every time, simplify testing operations and improve test efficiency. In S720, the separated dissolved organic halide may be converted to a dissolved inorganic halide using a photocatalysis technique which may not need the special high-temperature combustion equipment. Using the photocatalysis technique, the method may be performed at room temperature, and does not need to heat and take time to cool a sample, which may avoid the stringent environmental requirements of employing special high-temperature combustion equipment for conversion, and improve the test efficiency. It does not need to burn activated carbon and add a catalyst, which is more environmentally friendly. In S730, the converted dissolved inorganic halide may be analyzed using an ion meter to measure the content of the dissolved organic halide in the water to be measured.

According to another aspect of the present disclosure, a system for measurement of dissolved organic halide content in water may be provided. The system may use a method for measurement of dissolved organic halide content in water as described above. The system may include:

a separation device configured to separate DIH and DOH in a water sample to be measured using an electrodialysis technique;

a photocatalytic device configured to convert the separated DOH into DIH using a photocatalytic technique; and

an ion meter having an ion detector function configured to analyze the converted DIH and measure the content of DOH in the water sample to be measured.

As shown in FIG. 3, the separation device may include an electrodialyser including exchange membranes 100. The electrodialyser may include a first concentration chamber 200, a dilution chamber 210, and a second concentration chamber 220 arranged in order. The dilution chamber 210 may be connected with the first concentration chamber 200 and the second concentration chamber 220, respectively, via one or more exchange membranes 100. The first concentration chamber 200 may be configured with a first electrode 300. The second concentration chamber 220 may be configured with a second electrode 310. The polarity of an exchange membrane 100 connecting the first concentration chamber 200 and the dilution chamber 210 may be the same as the second electrode 310. The polarity of an exchange membrane 100 connecting the second concentration chamber 220 and the dilution chamber 210 may be the same as the first electrode 300. The first concentration chamber 200 and the second concentration chamber 220 may be configured to receive an injected inorganic electrolyte solution without halogens. The dilution chamber 210 may be configured to receive the injected water sample to be measured. The first electrode 300 and the second electrode 310 may be configured to generate an electric field after being powered. The electric field may drive inorganic halogen ions in the dilution chamber 210 to flow into the first concentration chamber 200 or the second concentration chamber 220 through the exchange membranes to separate the DIH and the DOH in the water sample to be measured.

Further, the first concentration chamber 200 may include a body, and a water outlet 400 and a water inlet 500 configured on the body of the first concentration chamber. The second concentration chamber 220 may include a body, and a water outlet 400 and a water inlet 500 configured on the body of the second concentration chamber 220.

Further, the separation device may further include an electrolyte solution adder connected to the water inlet 500. The electrolyte solution adder may be configured to add and/or inject an inorganic electrolyte solution to the first concentration chamber 200 and the second concentration chamber 220 according to a constant flow rate. A halogen ion concentration in the first concentration chamber 200 and the second concentration chamber 220 may be maintained by cooperatively adjusting the flow rate through the water outlet 400 and the flow rate through the water inlet 500.

Further, the photocatalytic device may include an ultraviolet irradiation device configured to convert the separated DOH into DIH using the photocatalytic technique.

The ultraviolet irradiation device may include at least one immersion ultraviolet lamp and/or at least one suspended irradiation ultraviolet lamp. The ultraviolet irradiation device may be configured to irradiate and converting the separated DOH obtained from the dilution chamber 210 into DIH. The at least one immersion ultraviolet lamp or at least one suspended irradiation ultraviolet lamp may include a low pressure mercury lamp, a medium pressure UV lamp, a high pressure mercury lamp, an amalgam UV lamp, an excimer excitation UV lamp, an xenon lamp, or a halide lamp.

Further, the content of the DOH in the water sample to be measured may be measured by analyzing the converted DIH using an ion meter. The benefits of the present disclosure include: the method and system for measuring the total content of dissolved organic halide in water may separate dissolved inorganic halide and dissolved organic halide in water to be measured using an electrodialysis technique, which may completely separate dissolved organic halide ions and dissolved inorganic halide ions, save resources, be environmentally friendly. The method does not involve replacing testing supplies every time, which may simplify testing operations and improve test efficiency. The method may convert the separated dissolved organic halide into dissolved inorganic halide using a photocatalytic technique which does not involve special high-temperature combustion equipment and may be easy to perform. And using the photocatalytic technique, the method may be performed at room temperature, and does not need to heat and take time to cool a sample, which may avoid the stringent environmental requirements of employing special high-temperature combustion equipment for conversion, and improve the test efficiency. And it does not need to burn activated carbon and add a catalyst, which is more environmentally friendly. And the method may analyze the converted dissolved inorganic halide using an instrument with an ion analysis function to analyze the content of dissolved organic halide in a water sample to be measured, which may achieve accurate measurement of the dissolved organic halide content in water, and the contents of fluorine, chlorine, bromine, and iodine in the dissolved organic halide.

It should be understood that the examples are merely illustrative of the present disclosure and are not a limitation of the embodiments of the present disclosure. Other variations or modifications of the various forms may be made by those skilled in the art based on the description. There is no need and no way to exhaust all of the implementations. The obvious changes or changes introduced by the principles of these to the present disclosure are still covered by the present disclosure.

What we claims:

1. A method for content measurement of dissolved organic halide in water, the method comprising:
separating a dissolved inorganic halide and a dissolved organic halide in a water sample to be measured using an electrodialysis technique;
converting the separated dissolved organic halide into a dissolved inorganic halide; and
measuring a content of the dissolved organic halide in the water sample to be measured by analyzing the converted dissolved inorganic halide using an ion analysis instrument.

2. The method of claim 1, wherein the separating the dissolved inorganic halide and the dissolved organic halide in the water sample to be measured using the electrodialysis technique includes:
providing an electrodialyser including a first concentration chamber, a dilution chamber, and a second concentration chamber arranged in order;
injecting an inorganic electrolyte solution without halogens into the first concentration chamber and the second concentration chamber;
injecting the water sample to be measured into the dilution chamber;
causing the electrodialyser to generate an electric field, the electric field being configured to drive inorganic halogen ions in the dilution chamber to flow into at least one of the first concentration chamber or the second concentration chamber; and
taking out the water sample including the dissolved organic halide from the dilution chamber to measure the content of the dissolved organic halide in the water sample to be measured.

3. The method of claim 2, wherein the converting the separated dissolved organic halide into a dissolved inorganic halide includes:
converting the dissolved organic halide in the water sample obtained from the dilution chamber into the dissolved inorganic halide by irradiating with ultraviolet (UV) light.

4. The method of claim 2, wherein the causing the electrodialyser to generate an electric field, includes:
applying a voltage to a first electrode and a second electrode of the electrodialyser, wherein:
the first electrode is configured in the first concentration chamber; and
the second electrode is configured in the second concentration chamber;
a polarity of an exchange membrane connecting the first concentration chamber and the dilution chamber is the same as the second electrode, and
a polarity of an exchange membrane connecting the second concentration chamber and the dilution chamber is the same as the first electrode.

5. The method of claim 2, wherein the dilution chamber is connected with the first concentration chamber and the second concentration chamber, respectively, via one or more exchange membranes.

6. The method of claim 1, wherein the converting the separated dissolved organic halide into a dissolved inorganic halide includes:
converting the separated dissolved organic halide to the dissolved inorganic halide, the conversion of the separated dissolved organic halide not involving a combustion of the separated dissolved organic halide.

7. The method of claim 1, wherein the converting the separated dissolved organic halide to a dissolved inorganic halide includes:
converting the separated dissolved organic halide to the dissolved inorganic halide at a temperature lower than a flash point of the separated dissolved organic halide.

8. The method of claim 1, wherein the converting the separated dissolved organic halide into a dissolved inorganic halide includes:
converting the separated dissolved organic halide into the dissolved inorganic halide at room temperature.

9. The method of claim 1, wherein the converting the separated dissolved organic halide into a dissolved inorganic halide includes:
converting the separated dissolved organic halide to a dissolved inorganic halide using a photocatalytic technique.

10. A system for measurement of dissolved organic halide content in water, comprising:
a separation device configured to separate a dissolved inorganic halide and a dissolved organic halide in a water sample to be measured using an electrodialysis technique;
a conversion device configured to convert the separated dissolved organic halide into a dissolved inorganic halide; and
an ion analysis device configured to measure the content of the dissolved organic halide in the water sample.

11. The system of claim 10, wherein
the separation device includes an electrodialyser including a first concentration chamber, a dilution chamber, and a second concentration chamber arranged in order, the dilution chamber being connected with the first concentration chamber and the second concentration chamber, respectively, via one or more exchange membranes,
the first concentration chamber includes a first electrode,
the second concentration chamber includes a second electrode,
the first concentration chamber and the second concentration chamber are configured to receive an injected inorganic electrolyte solution excluding halogens,
the dilution chamber is configured to receive the injected water sample to be measured, and
the first electrode and the second electrode are configured to generate, by applying a voltage, an electric field that drives the separated inorganic halogen ions in the dilution chamber to flow into the first concentration chamber or the second concentration chamber through the one or more exchange membranes.

12. The system of claim 11, wherein
the first concentration chamber includes a body, a water outlet and a water inlet configured on the body of the first concentration chamber;
the second concentration chamber includes a body, and a water outlet and a water inlet configured on the body of the second concentration chamber,
the water outlet and the water inlet of each of the first concentration chamber and the second concentration chamber are configured to maintain a halogen ion concentration in the first concentration chamber and the second concentration chamber by coordinately adjusting a flow rate through the water outlet and a flow rate through the water inlet.

13. The system of claim 11, wherein the separation device further comprises:
an electrolyte solution adder connected to the water inlet of each of the first concentration chamber and the second concentration chamber, the electrolyte solution adder being configured to inject an inorganic electrolyte solution to the first concentration chamber and the second concentration chamber, and coordinately adjusting a flow rate through the water outlet and a flow rate through the water inlet to maintain a halogen ion concentration in the first concentration chamber and the second concentration chamber.

14. The system of claim 11, wherein the exchange membranes are configured to separate the dissolved organic halide and the dissolved inorganic halide.

15. The system of claim 11, wherein the exchange membranes include at least one of an ion exchange membrane, a bipolar ion exchange membrane, a reverse osmosis membrane, a nanofiltration membrane, or a dialysis membrane.

16. The system of claim 10, wherein the conversion device is configured to convert the separated dissolved organic halide into the dissolved inorganic halide at room temperature.

17. The system of claim 10, wherein the conversion device includes a photocatalytic device configured to convert the separated dissolved organic halide into a dissolved inorganic halide using a photocatalytic technique.

18. The system of claim 17, wherein the photocatalytic device includes an ultraviolet irradiation device configured to irradiate the water sample obtained from the separation device to convert the dissolved organic halide in the water sample to the dissolved inorganic halide.

19. The system of claim 17, wherein the ultraviolet irradiation device includes at least one of a low pressure mercury lamp, a medium pressure UV lamp, a high pressure mercury lamp, an amalgam UV lamp, an excimer excitation UV lamp, an xenon lamp, or a halide lamp.

20. A method for content measurement of dissolved organic halide in water, the method comprising:
separating a dissolved inorganic halide and a dissolved organic halide in a water sample to be measured;
converting the separated dissolved organic halide to a dissolved inorganic halide using a photocatalytic technique; and
measuring a content of the dissolved organic halide in the water sample to be measured by analyzing the converted dissolved inorganic halide using an ion analysis instrument.
